(19) United States
(12) Patent Application Publication
(10) Pub. No.: US 2020/0197036 A1
(43) Pub. Date: Jun. 25, 2020

(54) JAWED SURGICAL INSTRUMENT COMPRISING A THROW LEVER SYSTEM

(71) Applicant: OLYMPUS WINTER & IBE GMBH, Hamburg (DE)

(72) Inventors: Hannes MIERSCH, Hamburg (DE); Andreas RÜHS, Ahrensburg (DE); Simon VOGT, Lübeck (DE)

(73) Assignees: OLYMPUS WINTER & IBE GMBH, Hamburg (DE); OLYMPUS WINTER & IBE GMBH, Hamburg (DE)

(21) Appl. No.: 16/321,415

(22) PCT Filed: Sep. 29, 2017
(86) PCT No.: PCT/EP2017/074850

§ 371 Date: Jan. 28, 2019

(30) Foreign Application Priority Data
Sep. 29, 2016 (DE) 10 2016 118 482.9

(51) Int. Cl. A61B 17/295

(52) U.S. Cl. CPC A61B 17/295; A61B 2017/2919

(57) ABSTRACT

A jawed surgical instrument has a jawed part on the distal end formed with two jaw branches, at least being pivotally supported. A handle actuating the jawed part is on the proximal end. The instrument includes a blade assembly reversibly displaceable in the axial direction for cutting tissue between branches, and a shaft designed as a tube. The jawed part and blade assemblies are actuatable by elements passing through the shaft. The jaw actuating element is longitudinally displaceable, from the handle, in the direction of the shaft. The blade actuating element is longitudinally displaceable in the direction of the shaft by a throw lever system. The throw lever system includes an actuating element, a coupling member, and a throw lever. The blade actuating element is longitudinally displaceable within the shaft in the shaft direction by actuating the actuating element.

### BACKGROUND

The invention relates to a jawed surgical instrument of the type stated in the preamble of claim 1.

Manual medical instruments of the generic type are generally used for laparoscopic procedures in the abdominal area of a patient. An end effector supported on a shaft is guided through an opening in the abdominal wall and into the abdominal cavity of the patient, and for manipulation of tissue and organs may be controlled at that location by operating a handle that is proximally supported on the shaft. During the surgical procedure, it is often necessary to sever tissue, using the end effector. For this purpose, the end effector may include a jawed part that is able to hold tissue between two jaw branches, and also a blade assembly that can cut the tissue held between the two jaw branches. To ensure sufficient flexibility of use during the surgical procedure, for the mentioned surgical cutting instruments it is also desirable for the jawed part to be flexibly rotatable about the axis of the instrument shaft.

Such surgical cutting instruments are described in U.S. Pat. No. 9,216,030 B2, US 2005/0107785 A1, and US 2004/0254573 A1, among other publications, and are sold as Ligasure Impact (Covidien), Enseal G2 Super Jaw (Ethicon), and HALO Cutting Forceps (OSTA). In these cutting instruments, a cutting blade is guided over the entire length of the jawed part, from the proximal end to the distal end. In the instruments, the cutting blade is moved along the shaft axis by various mechanical mechanisms on the handle side. The guide length of the operating mechanism on the handle side is a function of the jaw length over which the cutting blade is to be transported.

In addition, the instruments have a rotary wheel by means of which the jawed part can be rotated about the shaft axis. This rotation of the jawed part is desirable in order to further simplify the surgical procedure for the surgeon. The rotary wheel may be integrated into the handle, thus being operable with one hand, or may be mounted on the shaft, and thus being operable only with two hands. It is understood that the one-handed operability is clearly preferred in this case, since the surgeon thus has the other hand free, with which he/she can use other surgical instruments.

The mechanisms currently used on the handle side for guiding the cutting blade over the entire jaw length are very complex and costly to manufacture, or they require a large amount of space in the handle, so that simultaneously mounting the rotary wheel for jaw rotation in the handle is no longer possible. This is true in particular for very long jawed parts having lengths of greater than 30 mm, for example jawed parts having a length of 40 mm. Other mechanisms use a more space-saving gearwheel-based mechanism for driving the cutting blade. However, these are very complex and costly to manufacture.

The object of the present invention, therefore, is to provide a jawed surgical instrument having a blade assembly and a jawed part that is rotatable about the shaft axis, in which the guide mechanism for the cutting blade may be accommodated in the handle in a space-saving manner and has lower production costs. The aim of the space-saving accommodation in the handle part is to ensure that a rotary wheel for rotating the jawed part about the shaft axis may also be mounted on the handle part, thus enabling one-handed operation.

### DESCRIPTION OF THE INVENTION

This object is achieved by a jawed surgical instrument having the features of claim 1. In particular, the object is achieved by a jawed surgical instrument that includes a throw lever system, i.e., a system of levers that comprises at least one throw lever, as a guide mechanism for the cutting blade of a blade assembly.

In a first aspect, the invention relates to a jawed surgical instrument, on the distal end of which a jawed part that is formed with two jaw branches is situated, wherein at least one jaw branch is pivotally supported, and a handle for actuating the jawed part is situated on the proximal end of the jawed instrument, and the jawed electrosurgical instrument includes a blade assembly that is reversibly displaceable in the axial direction for cutting tissue between the jaw branches, and a shaft designed as a tube, on the distal end of which the jawed part is situated and on the proximal end of which the handle is situated, wherein the jawed part is actuatable by a jaw actuating element that passes through the shaft, and wherein the blade assembly is actuatable by a blade actuating element that passes through the shaft, and the jaw actuating element is longitudinally displaceable in the direction of the shaft via the handle situated on the proximal end of the shaft, characterized in that the blade actuating element is longitudinally displaceable in the direction of the shaft by means of a throw lever system, wherein the throw lever system includes an actuating element, a coupling member, and a throw lever, and wherein the blade actuating element is longitudinally displaceable within the shaft in the shaft direction by actuating the actuating element.

The jawed surgical instrument according to the invention has the advantage that the throw lever system may be manufactured in an advantageous manner and accommodated in a small space in the handle. It is thus possible for a rotary element, for example, for rotating the jawed part to likewise be mounted on the handle, thus allowing one-handed operation. At the same time, due to the use according to the invention of a throw lever system, a lift of the blade assembly of 40 mm or greater in the distal direction may be achieved. Thus, even very long jawed parts may be operated with one hand.

The jawed surgical instrument according to the invention may be used, for mechanically cutting bodily tissue. However, the jawed instrument may also be used for manipulating, for example holding, bodily tissue.

The “distal” end (of the jawed instrument or of the shaft, for example) in each case is the end that is generally farthest from the medical practitioner during use by same (i.e., the end that is closer to the patient during the surgical procedure). This corresponds to standard terminology in the field. Conversely, the “proximal” end in each case is the end that is generally closest to the medical practitioner during use by same (i.e., the end that is farther from the patient during the surgical procedure).

A jawed part that is designed with two jaw branches is situated at the distal end of the jawed surgical instrument. The jawed part according to the invention is designed with two jaw branches, at least one jaw branch being pivotably supported. The pivotable jaw branch is movable toward the second jaw branch, and in the closed state contacts same and/or is able to hold an object to be manipulated between the jaw branches. In one embodiment, the jawed part is designed with two jaw branches that are supported so as to be pivotable about an axis. The jaw branches are movable relative to one another, and in the closed state may contact one another and/or an object to be manipulated. In one embodiment, the jawed part as forceps or a gripper is designed with two movable jaw branches that are suitable for gripping and holding.

In addition, a handle for actuating the jawed part is situated at the proximal end of the jawed instrument according to the invention. The handle is preferably made up of two grip elements, each associated with a jaw branch. Alternatively, a first jaw branch may be associated with the shaft of the surgical instrument, and a second jaw branch may be associated with a grip element, for example by fastening to the actuating element, such as an actuating rod, that is connected to the grip element.

The jawed electrosurgical instrument includes a blade assembly, which is reversibly displaceable in the axial direction, for cutting tissue between the jaw branches. In the retracted state, the blade assembly may be concealed in the shaft of the jawed instrument.

The blade assembly may be reversibly displaced in the axial direction by the throw lever system of the jawed instrument. The throw lever system is thus a device for selectively and reversibly activating the blade assembly which is displaceable in the axial direction. During the displacement in the axial direction, the blade assembly generally slides through blade channels, provided that the tissue contact surfaces are close enough to one another. A blade channel may be provided in only one jaw branch or in both jaw branches, i.e., on or in the tissue contact surface of the jaw branch.

The tissue contact surface is the surface in each case of a jaw branch that is in direct contact with the tissue when tissue to be manipulated is held between the jaw branches. In other words, the tissue contact surface is the surface of the jaw branch that faces the second jaw branch. The term “surface” is not to be construed here as limiting, and in particular does not mean that the tissue contact surface is flat. Instead, the tissue contact surface of a jaw branch may have structures such as a blade channel and/or ribbing that improve the grip.

When the jaw branches grip tissue that is much thicker than the blade assembly and the diameter of the shaft or of the jawed part, so that the jaw branches do not come very close together, the blade assembly generally slides out of the shaft without being guided through the blade channels of the jaw branches. To avoid accidental cutting injuries, in one embodiment this situation may be prevented by means of a safety mechanism that ensures that the blade assembly is displaceable only in the axial direction, provided that the blade assembly is guided through at least one blade channel. Such safety mechanisms are known to experts in the field, and may include, for example, fastening of the blade assembly to one or both blade channels.

The blade assembly preferably includes at least one blade having at least one cutting edge in essentially the distal direction, i.e., at its distal end. The cutting edge of the blade may be beveled. The blade may be designed as a cutting plate.

The blade channel of a jaw branch and the complementary blade channel on the other jaw branch are preferably parts of a blade assembly. The blade channels extend through the centers of the jaw branches in such a way that a blade that is part of the blade assembly may be guided in the axial direction through one or both of the channels, from the proximal direction to the distal direction, and can cut tissue that is held between the two jaw branches. Suitable blade assemblies are described in US 2004/0254573 A1 and US 2005/0107785 A1, for example. The definitions and descriptions of the blade assemblies in these publications are therefore incorporated by reference herein.

In one embodiment, the jawed surgical instrument according to the invention is characterized in that it includes a shaft that is designed as a tube, on the distal end of which the jawed part is situated, and on the proximal end of which the handle is situated, and characterized in that the jawed part is actuatable by a jaw actuating element that passes through the shaft, and that is longitudinally displaceable, from the handle situated on the proximal end of the shaft, in the direction of the shaft. With the elongated shaft, such instruments are suited in particular for laparoscopic applications in the abdominal area, in which there is a relatively large distance between the surgeon and the surgical site. In one embodiment the jawed instrument is thus an endoscopic and/or laparoscopic instrument. With a shorter shaft, however, such instruments may also be used in open surgery. In one alternative embodiment, the instrument is therefore an instrument that is suitable for open surgery.

The jaw actuating element is preferably embodied as an actuating tube. This means that the jaw actuating element has an inner cavity in the longitudinal direction through which, for example, the blade actuating element described elsewhere herein may be guided.

The jaw actuating element is longitudinally displaceable from the handle, situated on the proximal end of the shaft, in the direction of the shaft. The longitudinal displacement is brought about by actuating the handle. The jawed part is opened or closed, i.e., moved into the open position or into the closed position, by the longitudinal displacement of the jaw actuating element.

The blade assembly is actuatable by a blade actuating element that passes through the shaft. The blade actuating element is preferably embodied as an actuating rod, and may extend, for example, in the interior of the jaw actuating element, which in this case is designed as an actuating tube.

The blade actuating element is longitudinally displaceable by actuating the actuating element within the shaft in the shaft direction. The longitudinal displacement is brought about by actuating the actuating element of the throw lever system. The blade assembly is displaced in the distal or proximal direction due to the longitudinal displacement of the blade actuating element. A displacement in the distal direction results in a cut in tissue that is held between the jaw branches.

The blade actuating element may preferably be displaced by a relatively large lift in the distal direction or in the longitudinal direction of the shaft. The blade actuating element is preferably displaceable in the longitudinal direction of the shaft by at least 30 mm, particularly preferably by at least 40 mm, by means of the actuating element. According to the invention, such a large lift with the simultaneous mounting of a rotary wheel for rotating the jawed part on the handle is achieved by using a throw lever system. Accordingly, the blade actuating element is displaceable in the longitudinal direction of the shaft, for example, by at least 30 mm, preferably by at least 40 mm, more preferably by at least 50 mm, most preferably by at least 60 mm. The blade actuating element may be displaceable, for example, in a range of 0 mm to 120 mm, preferably 0 mm to 90 mm, more preferably 0 mm to 60 mm, most preferably 0 mm to 45 mm.

Since the blade assembly is displaceable by the blade actuating element in the longitudinal direction of the shaft, the blade assembly is displaceable to the same extent as the blade actuating element.

The blade actuating element is longitudinally displaceable in the direction of the shaft by means of a throw lever system, the throw lever system including an actuating element, a coupling member, and a throw lever. The throw lever system is a coupling gear. Throw lever systems and throw lever gears are known to experts in the field, and are shown, for example, on the web site of the Digital Mechanism and Gear Library (http://www.dmg-lib.org).

The actuating element, coupling member, throw lever, and blade actuating element are motion-coupled in such a way that the movement of the actuating element by means of the coupling member and the throw lever moves the blade actuating element along the shaft in the longitudinal direction. For locking the blade assembly, it may be provided that the actuating element remains in a locked position and thus locks the blade assembly. The actuating element, coupling member, and throw lever are preferably movable in a shared movement plane or parallel movement planes, more preferably solely in this shared movement plane or these parallel movement planes. The actuating element, coupling member, and throw lever are each preferably essentially rigid elements, more preferably rigid, one-part elements.

The “actuating element” mentioned herein is a throw lever actuating element. The actuating movement of the actuating element provided for actuating the blade assembly or the blade actuating element preferably extends in parallel to the movement plane of the handle pieces. The actuating element may thus be easily operated with one finger of the same hand that also operates the handle pieces. To further facilitate the operation with one finger, the actuating element may have an ergonomic shape and/or surface that are/is spatially adapted to a finger. Thus, the actuating element may have a curvature, for example, in which the finger of the medical practitioner may be inserted, and/or ribbing of the surface that prevents the operating finger from slipping. The actuating element also has an elongated, preferably curved elongated, shape. The elongated actuating element preferably extends from a handle part of the jawed instrument in the distal, inferior, or distal-inferior direction.

As used herein, “inferior” refers to the direction beneath the shaft area during a normal use position. “Superior” refers to the direction above the shaft area during a normal act of use. In other words, the axis that extends through the movement plane of the handle from the inferior to the superior direction has an angle of 90° with respect to the longitudinal axis of the shaft.

The actuating element is preferably a second-class or third-class lever, preferably a third-class lever. A third-class lever, as is known, is a lever whose bearing point is situated at one end of the lever, and also in which the load to be overcome is situated farthest from the bearing point, while the force acts between the bearing point and the load. For a second-class lever, the bearing point is likewise situated at one end of the lever, but the load to be overcome acts between the bearing point and the force.

The point of the actuating element on which the load acts is formed by the connection between the actuating element and the coupling member. The actuating element is generally connected to the coupling member by means of a bearing that allows a rotational movement. The rotational movement is preferably parallel to the movement plane of the handle pieces, the same as the movement of the actuating element, the coupling member, and the throw lever. The rotational movement is preferably parallel to the shared movement plane of the actuating element, coupling member, and throw lever. In other words, the actuating element is connected to the coupling member by means of a radial bearing, for example. In addition, it is conceivable for the bearing to have additional degrees of freedom, for example a translational movement along the longitudinal axis of the actuating element or of the coupling member.

In one alternative embodiment, the connection between the actuating element and the coupling member may also have a rigid design. As a further alternative, the actuating element and the coupling member may be designed as a (rigid) part.

The coupling member connects the actuating element (throw lever actuating element) to the throw lever. The coupling member has an elongated shape, optionally an elongated curved shape. The coupling member extends essentially in parallel to the longitudinal axis of the shaft; this does not rule out that the longitudinal axis of the coupling member may be slightly tilted (+/−50°, for example) in the inferior or superior direction, for example, relative to an axis that is parallel to the shaft axis. The coupling member with its distal end is preferably connected to the actuating element (by means of the bearing described above), and with its proximal end is preferably connected to the throw lever (by means of the further bearing described elsewhere herein). The more distal end of the coupling member is preferably connected to the inferior end of the actuating element, and the more proximal end of the coupling member is preferably connected to the inferior end of the throw lever.

The coupling member is preferably connected only to the other components of the throw lever system, i.e., the actuating element, the throw lever, and the corresponding bearings and other bearing elements. This means that the coupling member is preferably not connected to parts of the jawed instrument that are not a part of the throw lever system.

The point on the coupling member on which the load acts is formed by the connection between the coupling member and the throw lever. The coupling member is connected to the throw lever by means of a bearing that allows a rotational movement. The rotational movement is preferably parallel to the movement plane of the handle pieces. The rotational movement is preferably parallel to the shared movement plane of the actuating element, the coupling member, and the throw lever. In other words, the coupling member is connected to the throw lever by means of a radial bearing, for example. In addition, it is conceivable for the bearing to have additional degrees of freedom, and for example to allow a translational movement along the longitudinal axis of the coupling member or of the throw lever.

The throw lever connects the coupling member to the blade actuating element. The throw lever has an elongated shape, optionally an elongated curved shape. The coupling member extends from the more proximal end of the coupling member, preferably in the superior or proximal-superior direction, to the proximal end of the blade actuating element.

The longitudinal axis of the throw lever thus preferably extends in the superior or proximal-superior direction (starting from the more proximal end of the coupling member).

The throw lever is preferably a first-class lever whose bearing point is situated between the acting force (via the coupling member) and the load (on the blade actuating element). The bearing point is preferably situated in the middle section of the throw lever, particularly preferably in the center of the throw lever. Alternatively, depending on the design and the required force and lift, the bearing point of the throw lever may be situated at some other position. Those skilled in the art are able to determine a suitable position for the bearing point of the throw lever, depending on the requirements.

The bearing point of the throw lever is formed by the fastening of the throw lever within the handle part. In one embodiment, the bearing point may be formed, for example, by a hollow cylinder in each of two plastic half-shells, the hollow cylinders being formed by an injection molding process, for example, and the two plastic shells forming the handle part. After the plastic half-shells are assembled, the ends of the two hollow cylinders are opposite one another, and could be joined, for example, by a pin that is inserted into the interior of the cylinders. The pin may then be used as a rotatable bearing point for the throw lever. For improving the stability, the pin may be made of a metallic material or may be a composite that includes a metallic material.

The load arm of the throw lever is preferably pivotable from −45° to +45° about a pivot axis that extends perpendicularly through its bearing point and orthogonally with respect to the longitudinal axis of the shaft. Extreme minimum forces in the start and end positions are thus avoided. The achievable cutting plate forces for a finger force of 30 N, for example, are initially approximately 14 N, increase to approximately 21 N, and then drop to 10 N.

The arrangement of the individual elements of the throw lever system ensures that the elements are functionally connected to one another in such a way that the blade actuating element may be moved in the longitudinal direction of the shaft (in both the distal and proximal directions) by actuating the actuating element (throw lever actuating element). The actuating element is thus functionally connected to the throw lever (and also to the blade actuating element) by means of the coupling member.

The throw lever is (functionally) connected to the proximal end area of the blade actuating element. In particular, the throw lever may be (functionally) connected to the proximal end area of the blade actuating element by means of a slide bearing.

The connection between the blade actuating element and the throw lever should preferably allow not only a rotational movement, but also a translational movement of the blade actuating element along the longitudinal axis of the throw lever. This ensures that the blade actuating element moves in a straight path in or parallel to the longitudinal axis of the shaft, and is not forced into a circular path. In one preferred embodiment, the blade actuating element thus has a throw lever connecting element on its proximal end that includes a journal area having a circular cross section and a thickened area proximal to the journal area, and the throw lever on its end that is connected to the blade actuating element has a slot, the journal area being guided through the slot in such a way that the thickened area is situated in the interior of the throw lever.

The throw lever may thus have a design that is tubular at least in sections, i.e., in the shape of a hollow cylinder, at least in the end area that is connected to the blade actuating element.

The jawed surgical instrument may also have a throw lever retraction element that automatically moves the end area of the throw lever, connected to the blade actuating element, in the proximal direction when the actuating element is released by the medical practitioner. Alternatively, the throw lever retraction element may be connected to the blade actuating element and may move it (and thus, also the end area of the throw lever that is connected to the blade actuating element) in the proximal direction. The throw lever retraction element may be a spring, for example.

The design described above allows space-saving accommodation of the throw lever system in the handle of the jawed instrument. The space-saving accommodation also allows the mounting of a rotary element for rotating the jawed part about the shaft axis on the handle, and thus, one-handed operation of the rotary element and the other functions of the handle (of the throw lever system, among others). In one preferred embodiment, the jawed part is thus rotatable about the longitudinal axis of the shaft by means of a rotary element situated in the proximal end area of the jawed surgical element. The jawed part is preferably rotatable about the longitudinal axis of the shaft in an angular range between 0° and 360°. The rotary element may be designed, for example, as a rotary wheel, such as a rotary wheel that is provided on the superior side of the jawed instrument.

In accordance with the above-stated advantages of the present invention, the rotary element and the actuating element are situated on or in the vicinity of the handle in such a way that one-handed operation of the grip elements of the handle, of the rotary element, and of the actuating element is made possible. This means that all of the above-mentioned elements may be operated with one hand, preferably without the medical practitioner having to release and/or grip the handle.

### BRIEF DESCRIPTION OF THE FIGURES

Exemplary embodiments of the invention are schematically illustrated in the drawings, which show the following:

FIG. 1 shows a side view of a jawed instrument according to the invention, with the blade assembly pushed between the jaw branches in the distal direction;

FIG. 2 shows a side view of a jawed instrument according to the invention, with the blade assembly moved back in the proximal direction, and thus for the most part being concealed in the shaft;

FIG. 3 shows a schematic illustration of the pivot area of the throw lever; and

FIG. 4 shows a schematic illustration of one embodiment of the connection between the throw lever and the blade actuating element.

### EXEMPLARY EMBODIMENTS

Further advantages, characteristics, and features of the present invention will become apparent from the following detailed description of exemplary embodiments with reference to the appended drawings. However, the invention is not limited to these exemplary embodiments.

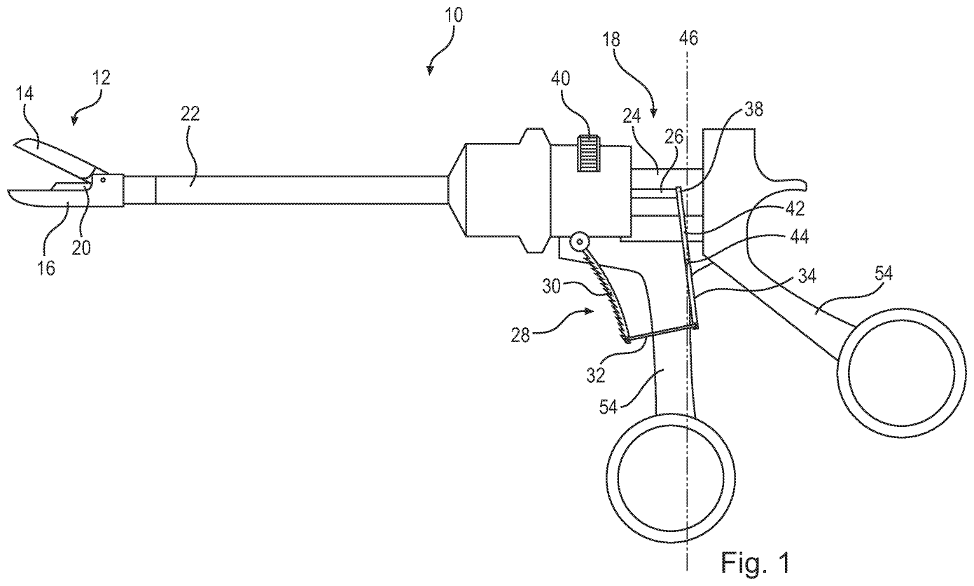

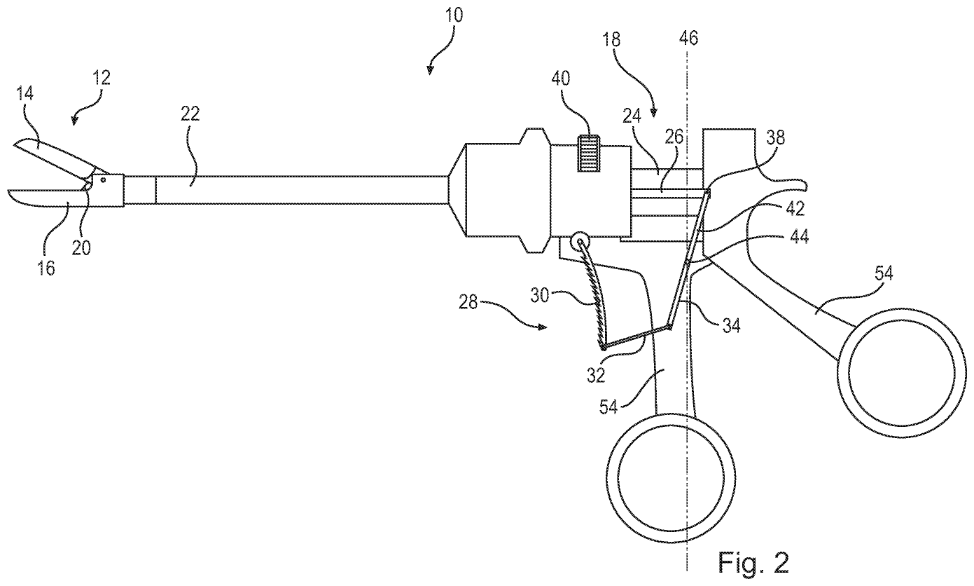

FIGS. 1 and 2 show side views of one embodiment of the jawed instrument 10 according to the invention; in FIG. 1 the blade assembly 20 is pushed between the two jaw branches 14, 16 in the distal direction, and in FIG. 2 the blade assembly 20 is retracted into the shaft 22 in the proximal direction. The blade assembly 20 is designed as a cutting plate. The position shown in FIG. 1 illustrates the process of cutting between the two jaw branches 14, 16. Tissue (not illustrated here) that is held between the two jaw branches 14, 16 is severed by the blade assembly 20. The jaw branches 14, 16 may both be closed. It may be provided in particular that the blade assembly 20 is displaceable between the two jaw branches 14, 16 only in the distal direction when the jaw branches 14, 16 are closed, i.e., their tissue contact surfaces are essentially in parallel with one another.

The jawed instrument 10 in FIGS. 1 and 2 has an elongated shaft 22, and is thus particularly suited for laparoscopic surgical procedures. In addition, the jawed instrument 10 has a handle 18 with a throw lever system 28 according to the invention.

The handle 18 has two grip elements 54, which may have different designs. Both or only one of the two grip elements 54 may be movable relative to the shaft 22.

In addition, the handle 18 includes a throw lever system 28 that is used for actuating the blade actuating element 26, and thus, the blade assembly 20. The throw lever system 28 includes an actuating element 30, a coupling member 32, and a throw lever 34. The actuating element 30 is fastened to the handle 18. The connection between the actuating element 30 and the handle 18 is established by a bearing that allows a rotational movement. The illustrated actuating element 30 has ribbing of its surface on a side facing the finger of the medical practitioner when the actuating element is actuated. Slipping of the finger is prevented in this way.

The actuating element 30 at its inferior end is connected to the coupling member 32 by means of a bearing that allows a rotational movement. The coupling member 32 connects the actuating element 30 to the throw lever 34. For this purpose, the connection between the more proximal end of the coupling member 32 and the inferior end of the throw lever 34 is also designed as a bearing that allows a rotational movement. Additional degrees of freedom for this connection are conceivable.

The throw lever 34 has a bearing point 44, which in the embodiment shown is designed as a bearing that allows a rotational movement. The pivot axis 46 extending perpendicularly through the bearing point 44 and orthogonally with respect to the longitudinal axis 23 of the shaft 22 extends through the bearing point 44 of the throw lever 34. The load arm 42 of the throw lever 34 is pivotable from −45° to +45° about the pivot axis 46.

The superior end of the throw lever 34 is connected to the proximal end of the blade actuating element 26 by means of a throw lever connecting element 38. The illustrated throw lever connecting element 38 is a bearing that allows a rotational movement and a translational movement. Alternatively, the superior end of the throw lever 34 may also be connected to the proximal end of the blade actuating element 26 via a simpler slide bearing 36. This embodiment is not illustrated.

The jawed instrument 10 in FIGS. 1 and 2 also includes a rotary element 40 that is mounted on the jawed instrument 10 in the superior direction from the actuating element 30. The rotary element 40 shown is designed as a rotary wheel, and has ribbing on its surface in order to simplify operation and in particular to prevent slipping.

FIG. 1 shows an actuating element 30 that is already actuated. It is apparent that the actuating element 30, in particular its inferior end, is pivoted in the proximal direction. In this way, the blade actuating element 26 and consequently the blade assembly 20 have been pushed between the jaw branches 14, 16 in the distal direction.

In FIG. 2 the actuating element 30 is relaxed and is displaced in the distal direction. In this way, the blade actuating element 26 and consequently the blade assembly 20 have been almost entirely retracted into the shaft 22 in the proximal direction.

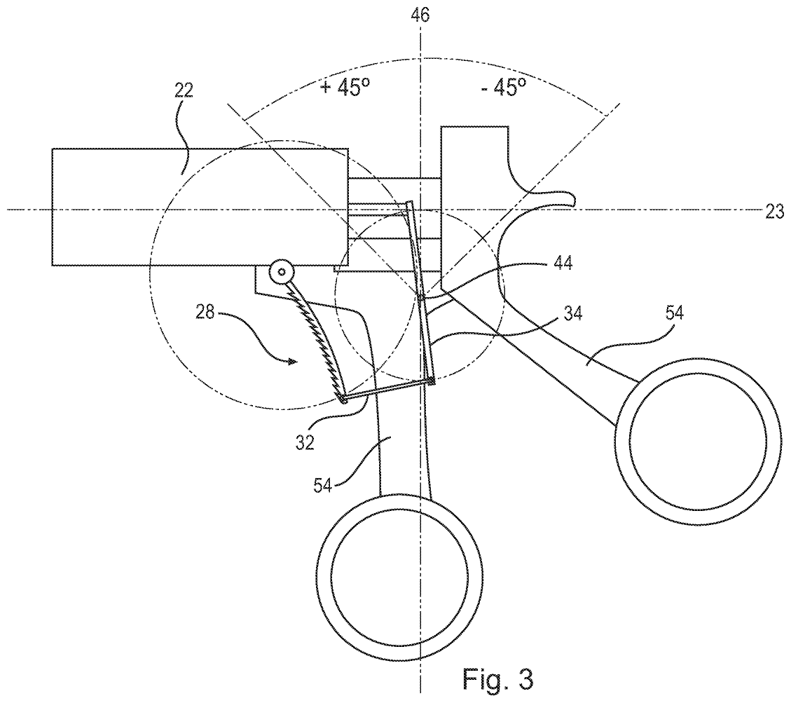

FIG. 3 shows a more detailed illustration of the pivot areas of the actuating element 30 and in particular of the throw lever 34. It is apparent that the actuating element 30 may be moved in a rotational movement around its fastening point on the handle 18. In the embodiment shown, the rotational movement of the actuating element 30 is in a plane parallel to the movement plane of the hand grips 54.

The throw lever 34 may likewise be moved in a rotational movement around its bearing point 44 (fastening point on the handle). In the embodiment shown, the rotational movement of the throw lever 34 is in a plane parallel to the movement plane of the hand grips 54. It is apparent that the load arm 42 of the throw lever 28 is pivotable from −45° to +45° about a pivot axis 46 that extends perpendicularly through its bearing point 44 and orthogonally with respect to the longitudinal axis 23 of the shaft 22.

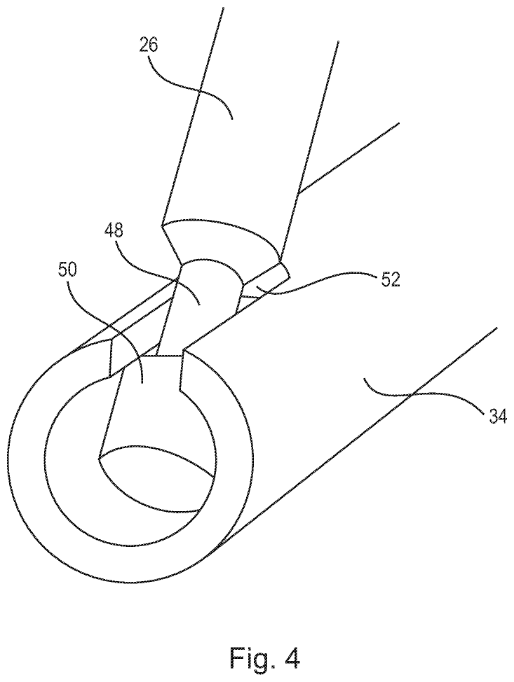

FIG. 4 shows a schematic illustration of one embodiment of the connection between the throw lever 34 and the blade actuating element 26. The illustrated blade actuating element 26 on its proximal end has a throw lever connecting element 38 that includes a journal area 48 having a circular cross section and a thickened area 50 proximal to the journal area 48. The throw lever 34, on its end that is connected to the blade actuating element 26, has a slot 52, the journal area 48 being guided through the slot 52 in such a way that the thickened area 50 is situated in the interior of the throw lever 34.

Although the present invention has been described above in detail with reference to the exemplary embodiments, it is naturally understood by those skilled in the art that the invention is not limited to these exemplary embodiments; rather, modifications are possible in such a way that individual features may be omitted or other combinations of the presented individual features may be implemented, provided that this does not represent a departure from the scope of protection of the appended claims. The present disclosure encompasses all combinations of the presented individual features.

### LIST OF REFERENCE NUMERALS

| | |
|---|---|
| 10 | jawed surgical instrument |
| 12 | jawed part |
| 14 | jaw branch |
| 16 | jaw branch |
| 18 | handle |
| 20 | blade assembly |
| 22 | shaft |
| 23 | shaft longitudinal axis |
| 24 | jaw actuating element |
| 26 | blade actuating element |
| 28 | throw lever system |
| 30 | actuating element |
| 32 | coupling member |
| 34 | throw lever |
| 36 | slide bearing |
| 38 | throw lever connecting element |
| 40 | rotary element |
| 42 | load arm of the throw lever |
| 44 | bearing point of the throw lever |
| 46 | pivot axis |
| 48 | journal area |
| 50 | thickened area |
| 52 | slot |
| 54 | grip element |

1. A jawed surgical instrument, on the distal end of which a jawed part that is formed with two jaw branches is situated, wherein at least one jaw branch is pivotally supported, and a handle for actuating the jawed part is situated on the proximal end of the jawed instrument, and the jawed electrosurgical instrument includes a blade assembly that is reversibly displaceable in the axial direction for cutting tissue between the jaw branches, and a shaft designed as a tube, on the distal end of which the jawed part is situated and on the proximal end of which the handle is situated, wherein the jawed part is actuatable by a jaw actuating element that passes through the shaft, and wherein the blade assembly is actuatable by a blade actuating element that passes through the shaft, and the jaw actuating element is longitudinally displaceable, from the handle situated on the proximal end of the shaft, in the direction of the shaft, wherein the blade actuating element is longitudinally displaceable in the direction of the shaft by means of a throw lever system, wherein the throw lever system includes an actuating element, a coupling member, and a throw lever, and wherein the blade actuating element is longitudinally displaceable within the shaft in the shaft direction by actuating the actuating element.

2. The jawed surgical instrument according to claim 1, wherein the actuating element is functionally connected to the throw lever by means of the coupling member.

3. The jawed surgical instrument according to claim 1, wherein the actuating element and the coupling member are joined by means of a bearing that allows a rotational movement, or the actuating element and the coupling member are joined by a rigid connection.

4. The jawed surgical instrument according to claim 1, wherein the throw lever is connected to the proximal end area of the blade actuating element.

5. The jawed surgical instrument according to claim 1, wherein the throw lever is connected to the proximal end area of the blade actuating element by means of a slide bearing.

6. The jawed surgical instrument according to claim 1, wherein the blade actuating element has a throw lever connecting element on its proximal end that includes a pin area having a circular cross section and a thickened area proximal to the pin area, and the throw lever on its end that is connected to the blade actuating element has a slot, the pin area being guided through the slot in such a way that the thickened area is situated in the interior of the throw lever.

7. The jawed surgical instrument according to claim 1, wherein the blade actuating element is displaceable in the longitudinal direction of the shaft by at least 30 mm by means of the actuating element.

8. The jawed surgical instrument according to claim 1, wherein the jawed part is rotatable around the longitudinal axis of the shaft by means of a rotary element situated in the proximal end area of the jawed surgical element.

9. The jawed surgical instrument according to claim 1, wherein the rotary element is designed as a rotary wheel.

10. The jawed surgical instrument according to claim 1, wherein the rotary element and the actuating element are situated on or in the vicinity of the handle in such a way that one-handed operation of the grip elements of the handle, of the rotary element, and of the actuating element is made possible.

11. The jawed surgical instrument according to claim 1, wherein the load arm of the throw lever is pivotable from −45° to +45° about a pivot axis that extends perpendicularly through its bearing point and orthogonally with respect to the longitudinal axis of the shaft.
